(19) United States
(12) Patent Application Publication
(10) Pub. No.: US 2025/0160834 A1
(43) Pub. Date: May 22, 2025

(54) SUTURABLE CUFF, METHOD OF INTEGRATING A TISSUE CONSTRUCT WITH A HOST ORGAN OR VASCULATURE, AND METHOD OF MAKING A SUTURABLE CUFF

(71) Applicant: President and Fellows of Harvard College, Cambridge, MA (US)

(72) Inventors: Daniel S. REYNOLDS, Cambridge, MA (US); Sebastien G.M. UZEL, Cambridge, MA (US); Jennifer A. LEWIS, Cambridge, MA (US); Ramsey DOOLITTLE, Cambridge, MA (US); Luba PERRY, Cambridge, MA (US)

(21) Appl. No.: 18/837,342

(22) PCT Filed: Feb. 21, 2023
(86) PCT No.: PCT/US2023/062916

§ 371 Date: Aug. 9, 2024

Related U.S. Application Data
(60) Provisional application No. 63/313,085, filed on Feb. 23, 2022.

(51) Int. Cl. A61B 17/11; A61B 17/00

(52) U.S. Cl. CPC A61B 17/11; A61B 2017/00004; A61B 2017/00526; A61B 2017/1107; A61B 2017/1132; A61B 2017/1135

(57) ABSTRACT

A suturable cuff for integrating a tissue construct in vivo with a host organ or vasculature comprises a hollow body having an anastomotic end, an anchoring end, and one or more lumens, where each lumen extends through the hollow body from a proximal opening at the anastomotic end to a distal opening at the anchoring end. The anastomotic end is configured for integration with one or more body vessels, and the anchoring end includes one or more anchoring features for connection with the tissue construct.

### RELATED APPLICATION

The present patent document claims the benefit of priority under 35 U.S.C. 119(e) to U.S. Provisional Patent Application No. 63/313,085, which was filed on Feb. 23, 2022, and is hereby incorporated by reference in its entirety.

### TECHNICAL FIELD

The present disclosure is related generally to biomedical devices and more particularly to a biomedical device for anastomosing engineered tissue and/or organ(s) to in vivo host vasculature.

### BACKGROUND

Despite advances in research and increased awareness of organ donation and transplantation, there continues to be a gap between the need for organs and available organ donations. Currently, more than 100,000 men, women and children are waiting for lifesaving organ transplants. Every ten minutes in the U.S., another person is added to the national transplant waiting list, and 7,000 deaths occur every year in the U.S. because organs are not donated in time. It would be beneficial to develop alternative technologies to enable organ transplantation to take place without waiting for donor organs.

The emerging ability to engineer three-dimensional (3D) vascularized tissues on demand may enable scientific and technological advances in tissue engineering, drug screening, toxicology, 3D tissue culture, organ repair, and organ transplantation. To produce 3D engineered tissue constructs that mimic natural tissues and, ultimately, organs, several key components—cells, extracellular matrix (ECM), and vasculature—may be assembled in complex arrangements. Each of these components plays a vital role: cells are the basic unit of all living systems, ECM provides structural support, and vascular networks provide efficient nutrient and waste transport, temperature regulation, delivery of factors, and long-range signaling routes. Without perfusable vasculature within a few hundred microns of each cell, three-dimensional tissues may quickly develop necrotic regions. Recently, progress has been made in embedding vascular networks in tissue constructs via 3D printing, as described for example in U.S. Pat. No. 10,117,969 to Lewis et al., entitled “Method of Printing a Tissue Construct with Embedded Vasculature,” which is hereby incorporated by reference. In order to successfully employ such vascularized tissue constructs for in vivo applications, it would be advantageous to develop a mechanism to integrate the tissue construct into a host organ or vasculature.

### BRIEF SUMMARY

A suturable cuff, a method of integrating a tissue construct with a host organ or vasculature, and a method of making the suturable cuff are described in this disclosure.

The suturable cuff comprises a hollow body having an anastomotic end, an anchoring end, and one or more lumens, where each lumen extends through the hollow body from a proximal opening at the anastomotic end to a distal opening at the anchoring end. The anastomotic end is configured for integration with one or more body vessels, and the anchoring end includes one or more anchoring features for connection with a tissue construct.

The method of integrating a tissue construct with a host organ or vasculature comprises: providing a suturable cuff comprising a hollow body having an anastomotic end, an anchoring end, and one or more lumens, each lumen extending through the hollow body from a proximal opening at the anastomotic end to a distal opening at the anchoring end; anchoring a tissue construct including a vascular channel to the anchoring end of the suturable cuff, the vascular channel being positioned to be in fluid communication with the distal opening(s); inserting the suturable cuff with anchored tissue construct into a body of a patient; anastomosing one or more body vessels to the anastomotic end of the suturable cuff, whereby the one or more body vessels are in fluid communication with the proximal opening(s); and perfusing the suturable cuff and the anchored tissue construct with blood or another bodily fluid from the patient.

A method of making a suturable cuff comprises: providing a mold having a cavity shaped to form an exterior of a hollow body, the cavity comprising an upper cavity portion and a lower cavity portion; filling the lower cavity portion with a precursor material; situating one or more anchoring features at an end of the lower cavity portion at a cavity location shaped to form part of an anchoring end of the hollow body, and situating one or more rods in the lower cavity portion at one or more cavity regions configured to define one or more lumens of the hollow body, wherein the precursor material in the lower cavity portion at least partly surrounds the one or more anchoring features and the one or more rods; placing the upper cavity portion over the lower cavity portion to close the mold; curing the precursor material to form a polymeric body surrounding the one or more rods and part of the one or more anchoring features; and opening the mold and removing the one or more rods, thereby forming a hollow body including the one or more anchoring features, and extracting the hollow body from the mold for use as a suturable cuff.

### BRIEF DESCRIPTION OF THE DRAWINGS

FIGS. 1A and 1B show examples of a suturable cuff.

FIG. 2 shows a tissue construct anchored to a suturable cuff; notably, a vascular channel within the tissue construct is in fluid communication with distal openings of the suturable cuff.

FIG. 3 schematically illustrates an exemplary path to obtain a perfusable tissue construct integrated with a suturable cuff for implantation in a patient.

FIGS. 4A-4D illustrate anchoring features of different geometries

FIGS. 4E-1-4E-3 show different views of a suturable cuff including anchoring features comprising rings and struts.

FIGS. 5A and 5B show views of an exemplary suturable cuff including one or more catheter liners contained within the hollow body.

FIG. 6 illustrates body vessels anastomosed to the anastomostic end of a suturable cuff via a side-to-end “T” connection.

FIGS. 7A-7C schematically show fabrication of a tissue construct and anchoring to a suturable cuff.

FIGS. 8A-8G show an exemplary tool and method for integrating a suturable cuff with a tissue construct

FIGS. 9A-9H illustrate a molding process to fabricate an exemplary suturable cuff.

### DETAILED DESCRIPTION

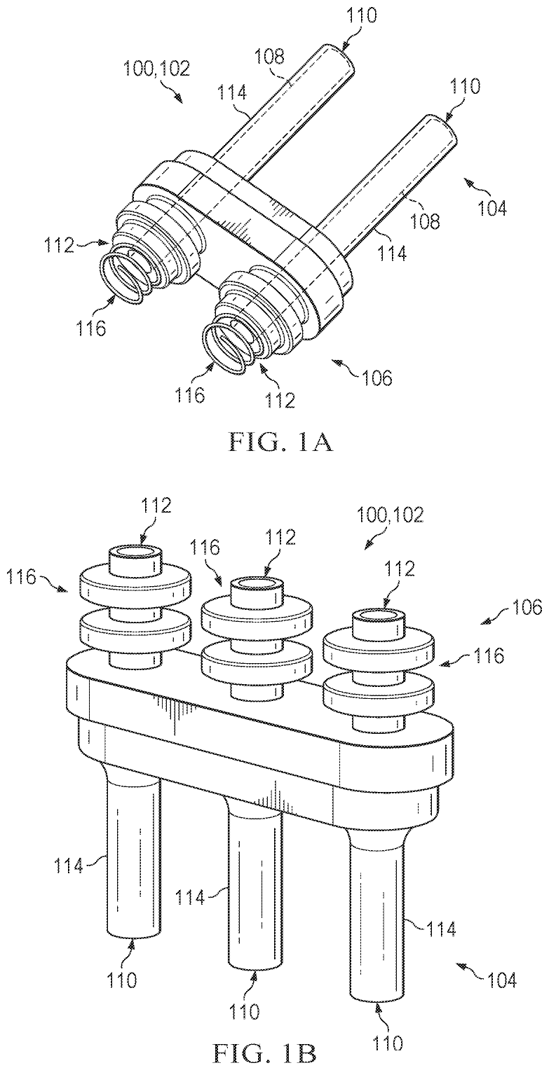

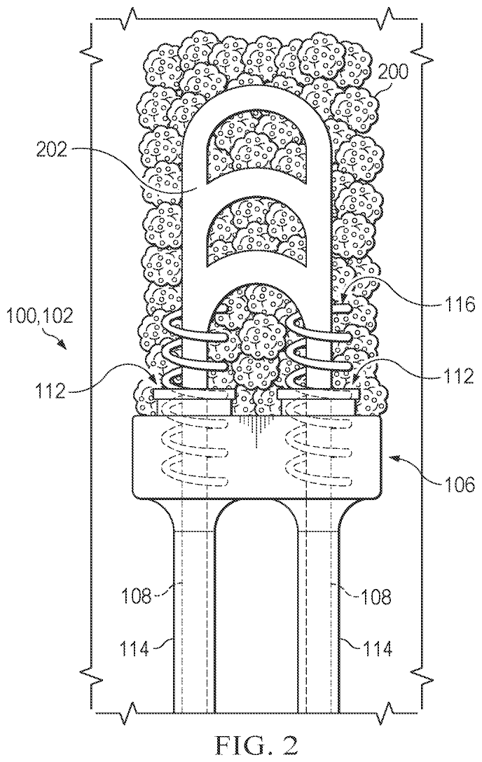

Referring to FIG. 1A, a suturable cuff 100 for connecting a tissue construct with a host organ or vasculature (“the host”) includes a hollow body 102 having an anastomotic end 104, an anchoring end 106, and one or more lumens 108 extending therebetween. The anastomotic end 104 is configured for connection with one or more body vessels (e.g., a vein, an artery, a lymphatic vessel, the bile duct or another duct, and/or a hollow organ such as the bladder, etc.) which may form part or all of the host organ or vasculature. The anchoring end 106 includes one or more anchoring features 116 for integration with a tissue construct 200, as discussed below in reference to FIG. 2. Each lumen 108 extends through the hollow body 102 from a proximal opening 110 at the anastomotic end 104 to a distal opening 112 at the anchoring end 106 for perfusion of blood or another bodily fluid from the host. In some examples, the suturable cuff 100 may include two (or more) lumens 108, as shown, and thus two (or more) proximal openings 110 at the anastomotic end 104 and two (or more) distal openings 112 at the anchoring end 106. Accordingly, in one example, the anastomotic end 104 may be joined to both a vein and an artery in vivo, and blood from the host vasculature may flow into and out of the tissue construct 200 joined to the suturable cuff 100 at the anchoring end 106. In other examples, the suturable cuff 100 may include three lumens 108, as shown in FIG. 1B, and thus may have three distal openings 112 and three proximal openings 110. In this embodiment, the anastomotic end 104 may be joined to a vein and artery for blood flow and to a third channel for transport of another bodily fluid. Typically, the suturable cuff 100 has a length in a range from about 2 mm to about 10 mm, as measured from the anchoring end 106 to the anastomotic end 104. Preferably, the suturable cuff 100 is suturable and hemocompatible, as discussed below, and is engineered for integration with the tissue construct 200.

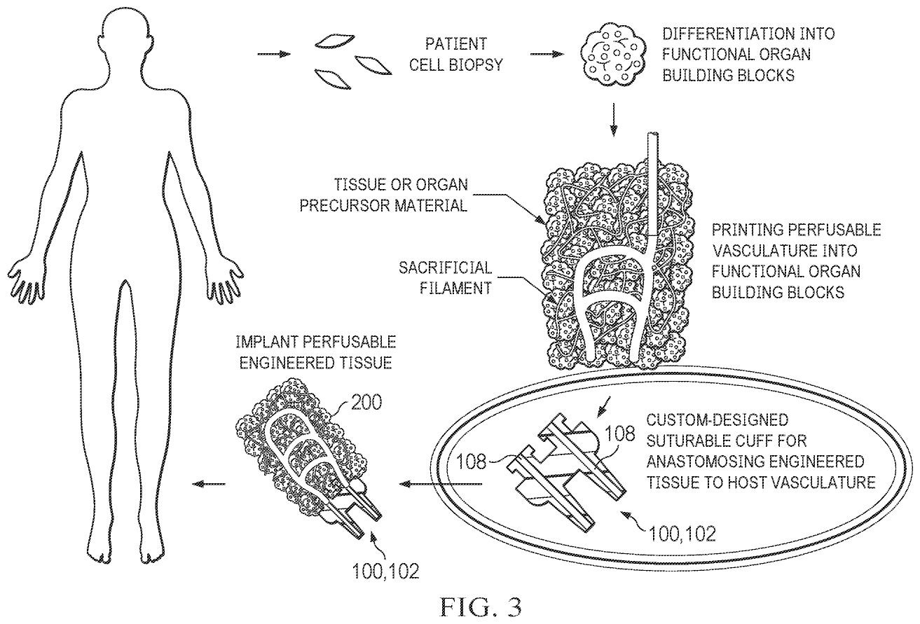

The term “tissue construct” may refer to any engineered tissue or organ that comprises cells and extracellular matrix material, preferably with interpenetrating vasculature. FIG. 2 is a schematic showing an exemplary tissue construct 200 including a vascular channel (or network of vascular channels) 202. The cells may be stem cells derived from a patient biopsy, as illustrated in FIG. 3. In some examples, the tissue construct may be a multi-layered tissue construct that includes from two to eight cell layers. The tissue construct 200 may have any size and geometry that may be prepared by 3D printing or another fabrication method, as discussed below.

At the anastomotic end 104, each of the lumens 108 is contained within a tubular or conical projection 114 from the hollow body 102, as shown in FIG. 1A, that facilitates connection with the host. Typically, each tubular or conical projection 114 has a wall thickness in a range from about 100 microns to about 1 mm. The wall thickness may be constant or variable along a length of each tubular or conical projection 114. The tubular or conical projection(s) 114 may be straight, curved, and/or angled in a desired direction. To facilitate in vivo attachment, the tubular or conical projection(s) 114 may be flexible. As discussed below, the one or more tubular or conical projections 114 may be sewn or sutured to part of the host organ or vasculature to permit perfusion of blood or another bodily fluid through the hollow body 102 and into the tissue construct 200.

In some examples, the tubular or connection projection(s) 114, as well as other portions or the entirety of the hollow body 102, may comprise a natural or synthetic polymer, such as an elastomer (e.g., silicone, or, more specifically, medical-grade silicone) and/or an extracellular matrix protein (e.g., collagen, fibrin, fibronectin, gelatin, and/or elastin). Advantageously, medical-grade silicone is commercially available in a range of stiffnesses. At a suitable stiffness, the silicone may exhibit a high tear strength, which ensures good suturability. Furthermore, the silicone surface may be modified if desired for hemocompatibility. The lumen 108 extending through the hollow body 102 may have a diameter in a range from about 100 microns to about 10 mm. The diameter may vary along a length of the lumen 108 or may be constant along the length, in which case the lumen 108 may have a cylindrical shape.

The lumen 108 and/or an outer surface of part or all of the hollow body 102 (e.g., the tubular or conical projection(s) 114) may further include a functional coating, which may in some examples may comprise a hydrophilic or zwitterionic polymer, such as polyethylene glycol, polyacrylamide, polyurethane, poly(hydroxyethyl methacrylamide), poly(methacryloyloxylethyl phosphorylcholine), poly(sulfobetaine methacrylate), and/or poly(sulfobetaine acrylamide). It is also contemplated that the functional coating may include an anti-coagulant drug (e.g., heparin). Also or alternatively, the functional coating may include an anti-inflammatory and/or anti-fibrotic agent to reduce inflammation and/or fibrosis in adjacent tissue. The anti-inflammatory and/or anti-fibrotic agent may also or alternatively elute from the hollow body 102. In some examples, the functional coating may be an endothelialized coating comprising an extracellular matrix protein (e.g., collagen or fibronectin) and endothelial cells.

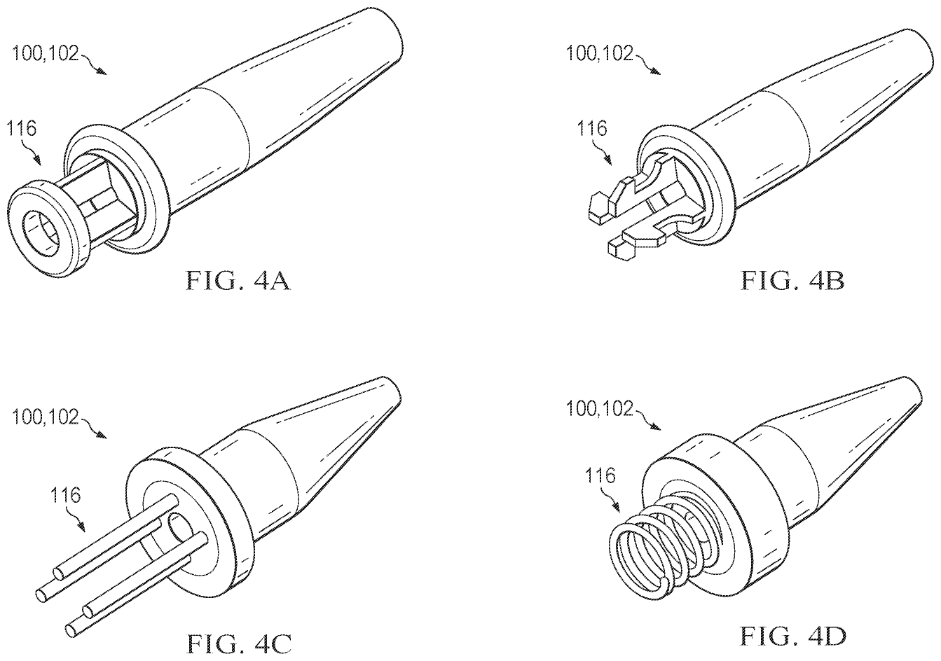

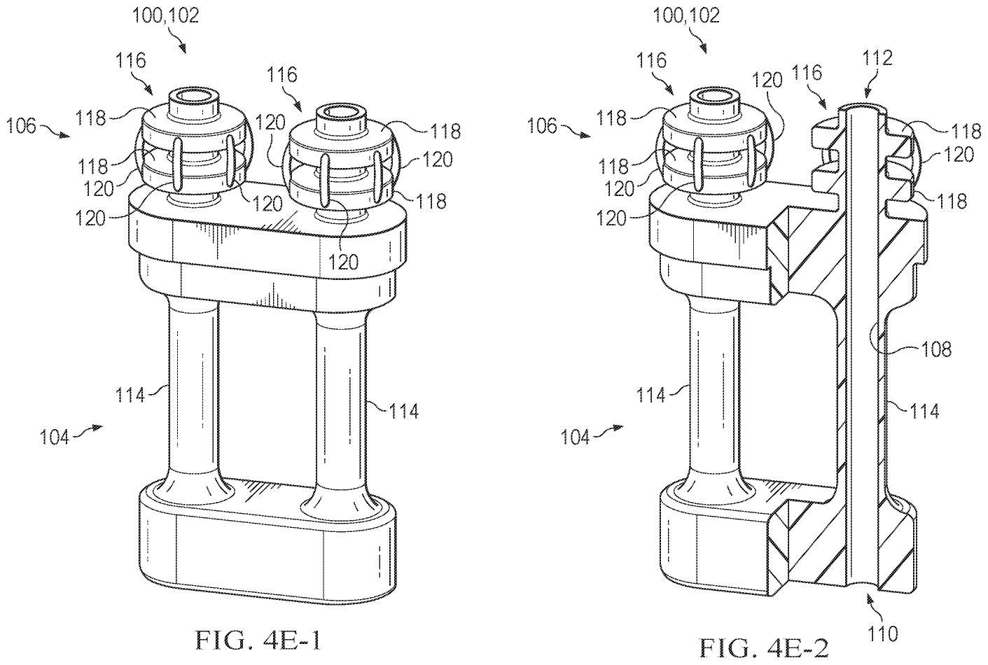

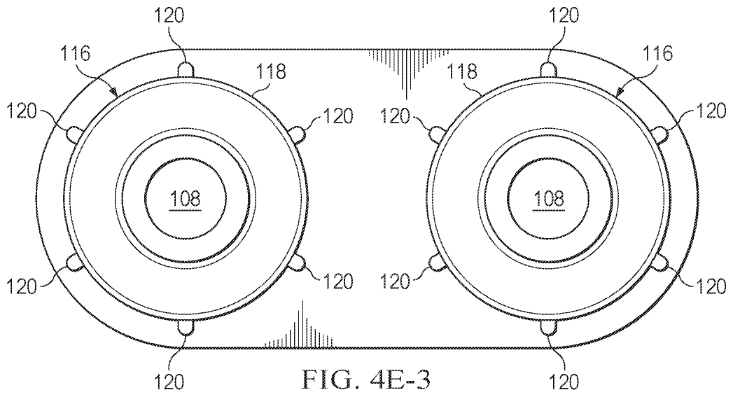

The one or more anchoring features 116 at the anchoring end 106 of the hollow body 102 are designed for engagement with the tissue construct 200. More specifically, the one or more anchoring features 116 are engineered for mechanical and/or chemical attachment (e.g., adhesion) to the tissue construct 200. In some examples, each anchoring feature 116 may comprise a hollow and/or porous structure configured to allow oxygen and nutrient exchange with the tissue construct. Referring to FIGS. 4A-4D, the one or more anchoring features 106 may take the form of one or more struts, one or more harpoons, one or more prongs, and/or one or more springs or coils, respectively. FIG. 4E-1 shows an embodiment in which the anchoring features 116 include one or more rings 118 which may optionally be connected by struts 120 (e.g., 3-8 struts spaced circumferentially apart; 6 are shown here). FIG. 4E-2 shows a partial cut-away view of the suturable cuff 100 and FIG. 4E-3 shows a top view of the suturable cuff 110. Also contemplated is an anchoring feature in the form of a mesh structure, e.g., a tubular or conical structure formed from a mesh. Each anchoring feature 116 has a shape and size configured for anchoring within the tissue construct 200. Preferably, while the tissue construct 200 is exposed to tensile and/or torsional forces within the host, the anchoring feature(s) 116 prevent the tissue construct 200 from being dislodged from the suturable cuff 100. Typically, each anchoring feature 116 has a length in a range from about 500 microns to about 5 mm, and/or a thickness in a range from about 50 microns to about 500 microns.

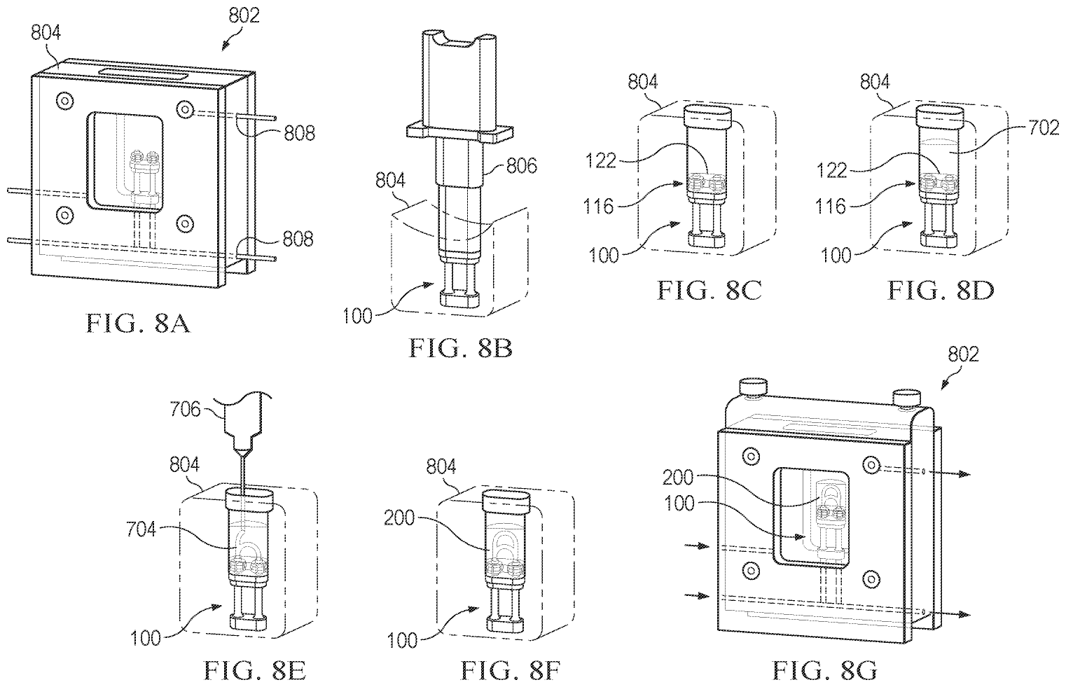

The anchoring feature(s) 116 may comprise a natural polymer, a synthetic polymer (e.g., polycaprolactone,poly-lactic-co-glycolic acid, or silicone (preferably medical-grade silicone)), and/or a metal or alloy (e.g., aluminum, chromium, cobalt, copper, gold, iron, magnesium, molybdenum, platinum, silver, tantalum, tin, titanium, zinc, zirconium, stainless steel, Ti—Al—V, Co—Cr, and/or Co—Cr—Mo). In some examples, the anchoring feature(s) 116 may be bioresorbable. As shown in FIG. 1A, the one or more anchoring features 116 may comprise a different material from the hollow body 102. Alternatively, the anchoring feature(s) and the hollow body 102 may comprise the same material. Accordingly, the suturable cuff 100 may have a multipiece and/or multimaterial construction in some examples, and in others the suturable cuff 100 may take the form of a monolithic body having a single-piece and/or single-material construction. To promote adhesion to the tissue construct, the anchoring feature(s) 116 may be coated with molecules that promote adsorption (e.g., polylactic acid, chitosan, and/or alginate) and/or molecules that promote covalent crosslinking (e.g., silanes functionalized with N-hydroxysuccinimide or aldehydes). In some examples, the suturable cuff 100 may further include a buffer hydrogel 122 positioned at the anchoring end 106, as can be seen in FIG. 8C, which is discussed below. The buffer hydrogel 122 may partly or fully encapsulate the one or more anchoring features 116 and may promote adhesion to the tissue construct 200. The buffer hydrogel is typically selected for compatibility with the extracellular matrix (ECM) material used for the tissue construct 200. For example, both the ECM material and the buffer hydrogel 122 may comprise the same hydrogel.

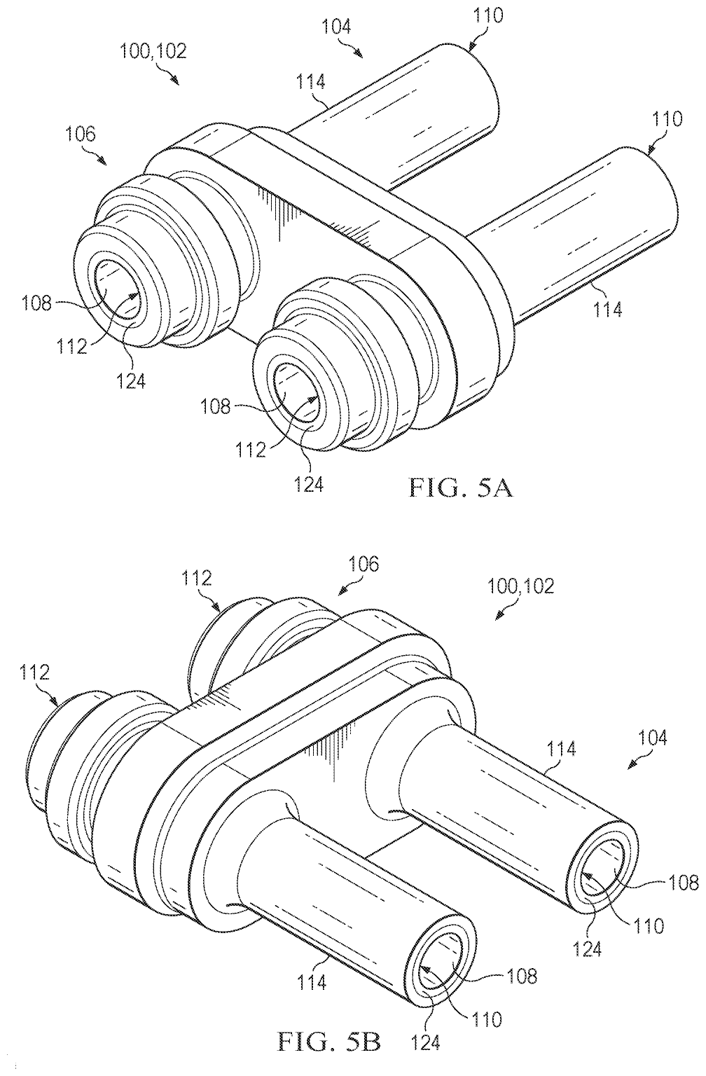

Referring to FIGS. 5A and 5B, the suturable cuff 100 may further include one or more catheter liners 124 contained within the hollow body 102, where each catheter liner 124 extends from the distal opening 112 to the proximal opening 110 and surrounds one of the lumens 108. Accordingly, when the suturable cuff 100 is in use, blood or other bodily fluids may contact the catheter liner 124 instead of the hollow body 102. There may be a one-to-one correspondence between the catheter liners 124 and the lumens 108. The catheter liner(s) 124 may be integrated with the hollow body 102 during fabrication (e.g., during molding, which is described below) or they may be inserted into the hollow body 102 after fabrication. In some examples, prior to integration with the hollow body, the one or more catheter liners may be etched on an outer surface thereof to promote adhesion to the hollow body.

The one or more catheter liners 124 may be formed from a polymer such as polytetrafluoroethylene (PTFE), expanded PTFE, polyethylene terephthalate, polyurethane, poly(glycolic acid), polylactic acid, and/or poly lactic-co-glycolic acid. The catheter liner(s) 124 may be endothelialized or chemically modified to provide desired functionality. For example, the one or more catheter liners 124 may include on inner surface(s) thereof a hydrophilic or zwitterionic polymer, such as polyethylene glycol, polyacrylamide, polyurethane, poly(hydroxyethyl methacrylamide), poly(methacryloyloxylethyl phosphorylcholine), poly(sulfobetaine methacrylate), and/or poly(sulfobetaine acrylamide). It is also contemplated that the catheter liner(s) 124 may include an anti-coagulant drug (e.g., heparin). Also or alternatively, the catheter liner(s) 124 may include an anti-inflammatory and/or anti-fibrotic agent to reduce inflammation and/or fibrosis in adjacent tissue. In some examples, the one or more catheter liners 124 may be endothelialized. For example, the one or more catheter liners 124 may include an extracellular matrix protein (e.g., collagen or fibronectin) and endothelial cells.

A method of connecting a tissue construct to a host organ or vasculature is now described in reference to FIGS. 2 and 3. The method includes providing a suturable cuff 100 having any of the features described in this disclosure. Generally speaking, the suturable cuff 100 comprises a hollow body 102 having an anastomotic end 104, an anchoring end 106, and one or more lumens 108, where each lumen 108 extends through the hollow body 102 from a proximal opening 110 at the anastomotic end 104 to a distal opening 112 at the anchoring end 106. In some examples, the suturable cuff 100 may include two (or more) lumens 108, and thus two (or more) proximal openings 110 at the anastomotic end 104 and two (or more) distal openings 112 at the anchoring end 106.

A tissue construct 200 that includes a vascular channel 202 is anchored to the anchoring end 106 of the suturable cuff 100. During the anchoring, which is described in greater detail below, the vascular channel 202 is positioned to be in fluid communication with the distal opening 112 of the hollow body 102, as illustrated in FIG. 2. If the suturable cuff 100 includes two or more lumens 108 and accordingly two or more distal openings 112 at the anchoring end 106, the vascular channel 202 may be positioned to be in fluid communication with the two or more distal openings 112, thereby facilitating, during in vivo use, fluid (e.g., blood) flow into and out of the tissue construct 200. It is noted that objects described in this disclosure as being “in fluid communication” with each other (e.g., the vascular channel 202 and the distal opening 112) are positioned with respect to each other such that a fluid can flow between and/or through the objects, in one or both directions.

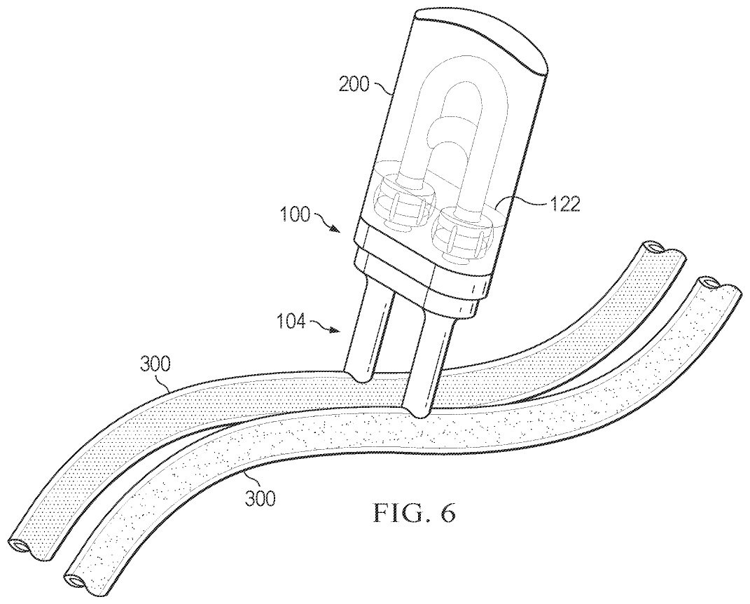

Referring now to FIG. 3, the suturable cuff 100 with the anchored tissue construct 200 is then inserted into a body of a patient and positioned at a desired in vivo location for connection to the host organ or vasculature. One or more body vessels (e.g., vein(s), artery (ies), duct(s), hollow organ(s) etc.) are sewn, sutured, or otherwise anastomosed to the anastomotic end 104 of the suturable cuff 100, such that the body vessel(s) are in fluid communication with the proximal opening(s) 110 of the hollow body 102. For example, the body vessel(s) 300 may be anastomosed to the anastomotic end 104 via an end-to-end connection or a side-to-end “T” connection, as shown in FIG. 6. As described above, the one or more body vessels may form part or all of the host organ or vasculature.

After anastomosis, the suturable cuff 100 and the anchored tissue construct 200 may be perfused with blood or another bodily fluid from the patient delivered through the body vessel(s). In experiments with adipose tissue, perfusion and patency of the suturable cuffs and the anchored tissue construct have been demonstrated. The above procedure may be used in various surgical applications such as reconstructive surgery and organ transplants (e.g., kidney transplants).

The anchoring or securing of the tissue construct 200 to the anchoring end 106 of the suturable cuff 100 may take place during or after fabrication of the tissue construct 200. As described above, the anchoring end 106 of the suturable cuff 100 includes one or more anchoring features 116, and thus the anchoring may be understood as partially or fully embedding the one or more anchoring features 116 within the tissue construct 200. In one example, the one or more anchoring features 116 may be inserted into the tissue construct 200 post-fabrication (of the tissue construct 200). Due to the configuration of the anchoring features 116, as illustrated according to several examples in FIGS. 4A-4E, the insertion may result in securing of the tissue construct to the suturable cuff 100. In addition, during the insertion, the suturable cuff 100 may be positioned such that the distal opening 112 of the hollow body 102 is in fluid communication with the vascular channel 202, as described above in reference to FIG. 2.

Alternatively, the anchoring may take place during fabrication of the tissue construct 200. In such an example, the tissue construct 200 may be formed around the anchoring feature(s) 116, such that the one or more anchoring features 116 are partially or fully embedded in the tissue construct 200. Preferably, during fabrication, a vascular channel 202 formed within the tissue construct 200 is positioned to be in fluid communication with the one or more distal openings 112 of the hollow body 102. For example, 3D printing or another fabrication method that allows for both embedding of the anchoring feature(s) 116 into the tissue construct 200 and alignment of the vascular channel(s) 202 with the distal opening(s) 112 may be employed, such that the tissue construct 200 is both anchored to the suturable cuff 100 and is perfusable after implantation into a patient's body.

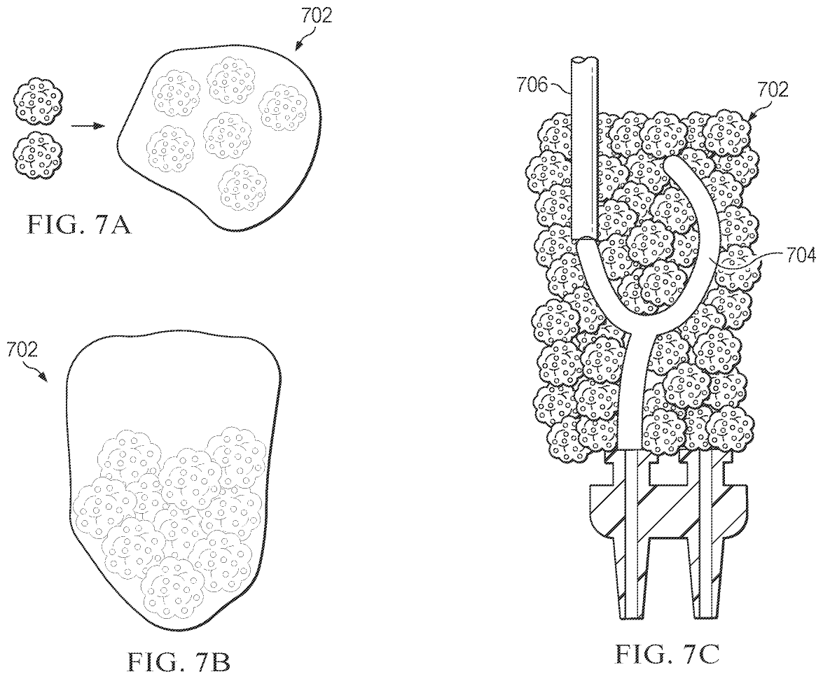

An exemplary 3D printing process that may be effective for anchoring the anchoring feature(s) 116 of the suturable cuff 100 within the perfusable tissue construct 200 during fabrication is described here in reference to FIGS. 7A-7C. The process may be referred to as sacrificial writing into functional tissue (SWIFT). The method begins (see FIG. 7A) with the preparation of a stem-cell derived tissue or organ precursor material 702, which may comprise stem cells mixed with an extracellular matrix solution. As illustrated in FIG. 3, stem cells obtained from a patient biopsy may be employed to form the tissue or organ precursor material.

The tissue or organ precursor material 702 may be compacted (e.g., via centrifugation) in a mold or other container to achieve high cellular density (e.g., up to or greater than about 200 million cells per milliliter), as illustrated in FIG. 7B. Advantageously, the tissue or organ precursor material 702 may have a rheology suitable to support embedded 3D printing, particularly when cooled to suitably low temperatures (e.g., 0-4° C.). The anchoring feature(s) 116 of the suturable cuff 100 may be added to the mold or other container in which printing takes place so as to be embedded in the tissue or organ precursor material. During embedded 3D printing, as illustrated in FIG. 7C, a nozzle 706 may be moved along a predetermined print path through the tissue or organ precursor material 702 while a filament 704 comprising a sacrificial ink is extruded through the printing nozzle 706 and deposited along the print path. Prior to printing, the precursor material 702 may be cooled to a temperature at or below about 4° C. to obtain the desired rheology, as indicated above. Advantageously, deposition of the filament 704 may be controlled such that the distal opening(s) 112 of the suturable cuff 100 are directly adjacent to part(s) of the filament 704; such alignment may facilitate obtaining a vascular channel in fluid communication with the distal opening(s) 112 after extraction of the sacrificial ink. After printing, the tissue or organ precursor material 702 may be heated to a suitable temperature (e.g., at least about 37° C.) at which the precursor material stiffens to form the tissue construct and the sacrificial ink liquifies to facilitate extraction. Thus, the sacrificial ink may be removed from the tissue construct and a vascular channel or network of vascular channels that are in fluid communication with the distal opening(s) 112 of the suturable cuff 100 may be formed. If desired, the vascular channel(s) may be seeded with endothelial cells to more closely mimic human blood vessels.

FIGS. 8A-8G show an exemplary tool and method for integrating a suturable cuff with a tissue construct. Referring to FIG. 8A, the suturable cuff 100 is mounted within the tool 802, which includes several sealable access ports 808. For stability during processing, the suturable cuff 100 may be attached to a stabilization platform connecting the tubular projections at the anastomotic end. In a first step, as shown in FIG. 8B, gelatin 804 may be introduced into the tool and poured around the cuff and a 3D printed mold 806, which may then be removed, leaving behind a reservoir. A high concentration fibrin gel or another hydrogel may be injected into the reservoir around the anchoring features, as shown in FIG. 8C, to form a buffer hydrogel 122. A tissue or organ precursor material 702 prepared as described above may be added into the reservoir over the hydrogel 118, as illustrated in FIG. 8D. Vascular channels may be templated using the SWIFT technology referred to above, as shown in FIG. 8E, where a nozzle 706 is moved along a predetermined print path through the tissue or organ precursor material while a filament 704 comprising a sacrificial ink is extruded through the nozzle 706. As shown, deposition of the filament 704 may be controlled such that the distal openings of the suturable cuff 100 are directly adjacent to ends of the filament 704. Upon heating, the sacrificial ink forming the printed filament may be liquified for removal and the tissue or organ precursor material may stiffen or cure to form the tissue construct, as illustrated in FIG. 8F. Finally, the sacrificial gelatin 804 may be rinsed away and the tissue construct 200 may be perfused with a culture medium, as illustrated in FIG. 8G.

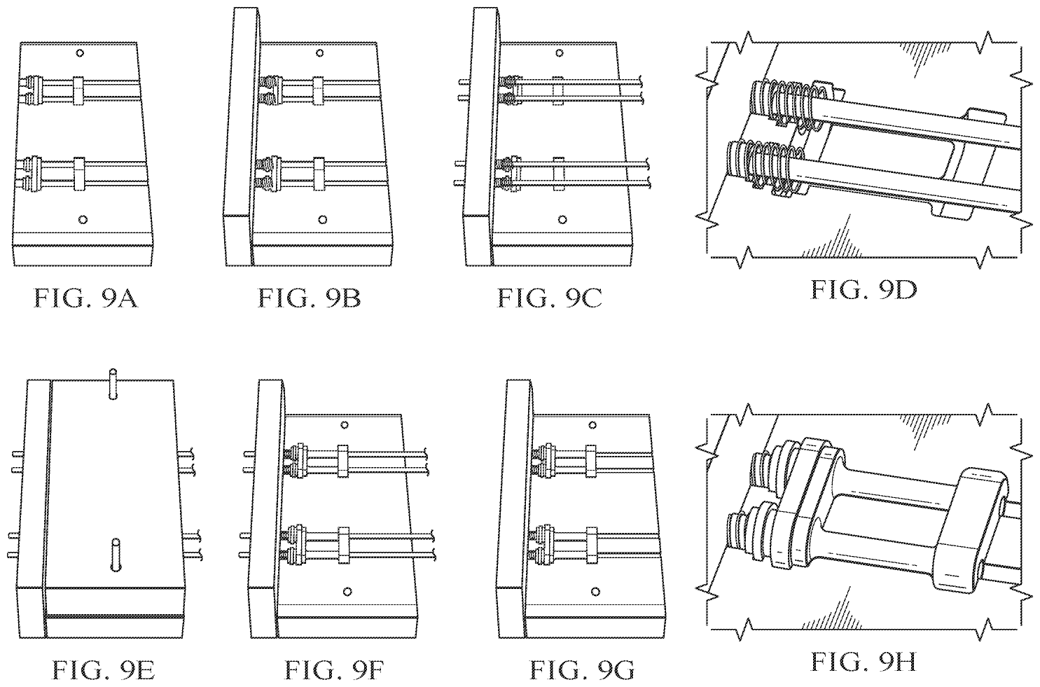

Also described in this disclosure is a method of making the suturable cuff. In one example, the suturable cuff may be made in a molding process. Alternatively, the suturable cuff may be fabricated by 3D printing. A description of an exemplary molding process is described in reference to FIGS. 9A-9H. The method includes providing a mold, which may be a two-piece mold as illustrated, having a cavity shaped to form an exterior of the hollow body. The cavity includes an upper cavity portion and a lower cavity portion. To fabricate the suturable cuff, the lower cavity portion is filled with a precursor material, e.g., a precursor material for a natural or synthetic polymer, such as an uncured polymer resin, as shown in FIG. 9A. Next, one or more anchoring features are situated at an end of the lower cavity portion at a cavity location shaped to form part of the anchoring end of the hollow body, as shown in FIG. 9B, and one or more rods (or pins), typically one or more metal rods or pins, are situated in the lower cavity portion at one or more cavity regions configured to define the lumen(s) of the hollow body, as shown in FIG. 9C, with a close-up view in FIG. 9D. The precursor material in the lower cavity portion at least partly surrounds the one or more anchoring features and the one or more rods. The size and shape of the rod(s) may be selected to determine the size and shape of the lumen(s) formed in the hollow body. An additional amount of the precursor material may be added to cover the one or more rods and may be added to the upper cavity portion. The upper cavity portion is set in place to close the mold, as shown in FIG. 9E, and the precursor material is cured to form a polymeric body surrounding the one or more rods and part of the anchoring feature(s), where the polymeric body comprises the desired natural or synthetic polymer (e.g., silicone). After curing, the mold is opened, as shown in FIG. 9F, the rods are removed, as shown in FIG. 9G, and a hollow body (close-up view in FIG. 9H) including the one or more anchoring features may be extracted for integration with a tissue construct and ultimately for in vivo use as a suturable cuff, as described above. It is noted in reference to FIG. 9H that the bar connecting the tubular projections at the anastomotic end of the suturable cuff may be useful as a support or stabilization platform during 3D printing (of the tissue construct) but is typically removed prior to implantation into a patient. It is also noted that, in an example in which the one or more anchoring features and the hollow body of the suturable cuff comprise the same material, the anchoring features may be formed by molding along with the hollow body. In such an example, the cavity of the mold may be shaped to form the exterior of the hollow body, as described above, and also the exterior of the anchoring feature(s).

The subject matter of this disclosure may relate to, among others, the following aspects:

A first aspect relates to a suturable cuff for integrating a tissue construct in vivo with a host organ or vasculature, the suturable cuff comprising: a hollow body having an anastomotic end, an anchoring end, and one or more lumens, each lumen extending through the hollow body from a proximal opening at the anastomotic end to a distal opening at the anchoring end, the anastomotic end being configured for integration with one or more body vessels, and the anchoring end including one or more anchoring features for connection with a tissue construct.

A second aspect relates to the suturable cuff of the preceding aspect, wherein the one or more anchoring features are configured for mechanical and/or chemical attachment to the tissue construct.

A third aspect relates to the suturable cuff of any preceding aspect, wherein each of the one or more anchoring features comprises a hollow and/or porous structure configured to allow oxygen and nutrient exchange with the tissue construct.

A fourth aspect relates to the suturable cuff of any preceding aspect, wherein the one or more anchoring features include one or more springs or coils, one or more harpoons, one or more prongs, one or more struts, one or more rings, and/or one or more mesh structures.

A fifth aspect relates to the suturable cuff of any preceding aspect, wherein each of the one or more anchoring features includes two or more rings connected by struts.

A sixth aspect relates to the suturable cuff of any preceding aspect, wherein the one or more anchoring features comprise a natural polymer, a synthetic polymer, and/or a metal or alloy

A seventh aspect relates to the suturable cuff of any preceding aspect, wherein the one or more anchoring features are bioresorbable.

An eighth aspect relates to the suturable cuff of any preceding aspect, wherein the one or more anchoring features are coated with one or more molecules to promote adhesion to the tissue construct, the one or more molecules being selected from the group consisting of: polylactic acid, chitosan, alginate, silanes functionalized with N-hydroxysuccinimide, and aldehydes.

A ninth aspect relates to the suturable cuff of any preceding aspect, further comprising a buffer hydrogel positioned at the anchoring end to promote adhesion to the tissue construct.

A tenth aspect relates to the suturable cuff of any preceding aspect, wherein each anchoring feature has a length in a range from about 500 microns to about 5 mm.

An eleventh aspect relates to the suturable cuff of any preceding aspect, wherein each anchoring feature has a thickness in a range from about 50 microns to about 500 microns.

A twelfth aspect relates to the suturable cuff of any preceding aspect, wherein the hollow body includes two lumens.

A thirteenth aspect relates to the suturable cuff of any preceding aspect, wherein the hollow body includes three lumens.

A fourteenth aspect relates to the suturable cuff of any preceding aspect, wherein the hollow body comprises a natural or synthetic polymer.

A fifteenth aspect relates to the suturable cuff of any preceding aspect, wherein the hollow body and/or the one or more anchoring features comprises an elastomer, such as silicone.

A sixteenth aspect relates to the suturable cuff of any preceding aspect, wherein the hollow body comprises an extracellular matrix protein or material selected from the group consisting of collagen, fibrin, fibronectin, gelatin, and elastin.

A seventeenth aspect relates to the suturable cuff of any preceding aspect, further comprising one or more catheter liners contained within the hollow body, each catheter liner extending from the distal opening to the proximal opening of the hollow body and surrounding one of the lumens.

An eighteenth aspect relates to the suturable cuff of the preceding aspect, wherein the one or more catheter liners comprise polytetrafluoroethylene (PTFE), expanded PTFE, polyethylene terephthalate, polyurethane, poly(glycolic acid), polylactic acid, and/or poly lactic-co-glycolic acid.

A nineteenth aspect relates to the suturable cuff of any preceding aspect, wherein the one or more catheter liners include on inner surface(s) thereof a hydrophilic or zwitterionic polymer.

A twentieth aspect relates to the suturable cuff of any preceding aspect, wherein the one or more catheter liners include on inner surface(s) thereof an anti-coagulant drug.

A twenty-first aspect relates to the suturable cuff of any preceding aspect, wherein the one or more catheter liners include on inner surface(s) thereof an anti-inflammatory and/or anti-fibrotic agent to reduce inflammation and/or fibrosis in adjacent tissue.

A twenty-second aspect relates to the suturable cuff of any preceding aspect, wherein the one or more catheter liners include on inner surface(s) thereof an extracellular matrix protein or material and endothelial cells, the one or more catheter liners being endothelialized.

A twenty-third aspect relates to the suturable cuff of any preceding aspect, wherein, prior to integration with the hollow body, the one or more catheter liners are etched on outer surface(s) thereof to promote adhesion to the hollow body.

A twenty-fourth aspect relates to the suturable cuff of any preceding aspect, wherein the anastomotic end of the hollow body includes proximal tubular or conical projections, and wherein each lumen is contained within one of the proximal tubular or conical projections.

A twenty-fifth aspect relates to the suturable cuff of any preceding aspect, wherein a wall thickness of each tubular or conical projection is in a range from about 100 microns to about 1 mm.

A twenty-sixth aspect relates to the suturable cuff of any preceding aspect, wherein each tubular or conical projection is straight, curved, and/or angled in a predetermined direction.

A twenty-seventh aspect relates to the suturable cuff of any preceding aspect, wherein each tubular or conical projection is flexible.

A twenty-eighth aspect relates to the suturable cuff of any preceding aspect, wherein each lumen has a diameter in a range from about 100 microns to about 10 mm.

A twenty-ninth aspect relates to the suturable cuff of any preceding aspect, wherein the diameter is constant along a length of the respective lumen.

A thirtieth aspect relates to the suturable cuff of any preceding aspect, wherein the diameter varies along a length of the respective lumen.

A thirty-first aspect relates to the suturable cuff of any preceding aspect, wherein the one or more lumens and/or an outer surface of the hollow body includes a functional coating.

A thirty-second aspect relates to the suturable cuff of any preceding aspect, wherein the functional coating comprises a hydrophilic or zwitterionic polymer.

A thirty-third aspect relates to the suturable cuff of any preceding aspect, wherein the functional coating includes an anti-coagulant drug.

A thirty-fourth aspect relates to the suturable cuff of any preceding aspect, wherein the functional coating includes an anti-inflammatory and/or anti-fibrotic agent to reduce inflammation and/or fibrosis in adjacent tissue.

A thirty-fifth aspect relates to the suturable cuff of any preceding aspect, wherein the functional coating comprises an extracellular matrix protein or material and endothelial cells, the functional coating being an endothelialized coating.

A thirty-sixth aspect relates to the suturable cuff of any preceding aspect, having a length from the anchoring end to the anastomotic end in a range from about 2 mm to about 10 mm.

A thirty-seventh aspect relates to a method of integrating a tissue construct with a host organ or vasculature, the method comprising: providing a suturable cuff comprising a hollow body having an anastomotic end, an anchoring end, and one or more lumens, each lumen extending through the hollow body from a proximal opening at the anastomotic end to a distal opening at the anchoring end; anchoring a tissue construct including a vascular channel to the anchoring end of the suturable cuff, the vascular channel being positioned to be in fluid communication with the distal opening(s); inserting the suturable cuff with anchored tissue construct into a body of a patient; anastomosing one or more body vessels to the anastomotic end of the suturable cuff, whereby the one or more body vessels are in fluid communication with the proximal opening(s); and perfusing the suturable cuff and the anchored tissue construct with blood or another bodily fluid from the patient.

A thirty-eighth aspect relates to the method of the preceding aspect, wherein the suturable cuff and the anchored tissue construct are leak-free.

A thirty-ninth aspect relates to the method of the preceding aspect, wherein the anchoring end of the suturable cuff includes one or more anchoring features, and wherein anchoring the tissue construct to the anchoring end of the suturable cuff comprises embedding the one or more anchoring features into the tissue construct.

A fortieth aspect relates to the method of any preceding aspect, wherein embedding the one or more anchoring features within the tissue construct comprises: preparing a tissue or organ precursor material comprising stem cells mixed with an extracellular matrix solution; embedding the one or more anchoring features at the anchoring end of the suturable cuff into the tissue or organ precursor material; moving a nozzle along a print path through the tissue or organ precursor material while a filament comprising a sacrificial ink is extruded through the printing nozzle and deposited along the print path; after deposition of the filament, heating the tissue or organ precursor material to a temperature at which the precursor material stiffens to form the tissue construct and the sacrificial ink liquifies for extraction; and removing the sacrificial ink from the tissue construct to form, within the tissue construct, a vascular channel in fluid communication with the distal opening(s) of the suturable cuff.

A forty-first aspect relates to the method of any preceding aspect, the method further comprising, before embedding the anchoring end of the suturable cuff into the tissue or organ precursor material, compacting the tissue or organ precursor material to increase cellular density.

A forty-second aspect relates to the method of any preceding aspect, further comprising, before moving the nozzle through the tissue or organ precursor material, cooling the tissue or organ precursor material to a temperature at or below 4° C.

A forty-third aspect relates to the method of any preceding aspect, wherein the print path is configured such that each distal opening of the suturable cuff is directly adjacent to a part of the filament.

A forty-fourth aspect relates to the method of any preceding aspect, wherein the temperature to which the tissue or organ precursor material is heated is at least about 37° C.

A forty-fifth aspect relates to the method of any preceding aspect, further comprising seeding the vascular channel with endothelial cells.

A forty-sixth aspect relates to the method of any preceding aspect, wherein embedding the one or more anchoring features into the tissue construct comprises inserting the one or more anchoring features into the tissue construct after fabrication thereof.

A forty-seventh aspect relates to the method of any preceding aspect, wherein anastomosing the body vessel to the anastomotic end comprises suturing the body vessel to the anastomotic end via an end-to-end connection or a side-to-end T connection.

A forty-eighth aspect relates to a method of making a suturable cuff, the method comprising: providing a mold having a cavity shaped to form an exterior of a hollow body, the cavity comprising an upper cavity portion and a lower cavity portion; filling the lower cavity portion with a precursor material; situating one or more anchoring features at an end of the lower cavity portion at a cavity location shaped to form part of an anchoring end of the hollow body; situating one or more rods in the lower cavity portion at one or more cavity regions configured to define one or more lumens of the hollow body, wherein the precursor material in the lower cavity portion at least partly surrounds the one or more anchoring features and the one or more rods; placing the upper cavity portion over the lower cavity portion to close the mold; curing the precursor material to form a polymeric body surrounding the one or more rods and part of the one or more anchoring features; andopening the mold and removing the one or more rods, thereby forming a hollow body comprising the one or more anchoring features, and extracting the hollow body from the mold for use as a suturable cuff.

To clarify the use of and to hereby provide notice to the public, the phrases “at least one of <A>, <B>, . . . and <N>” or “at least one of <A>, <B>, . . . or <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

While various embodiments have been described, it will be apparent to those of ordinary skill in the art that many more embodiments and implementations are possible. Accordingly, the embodiments described herein are examples, not the only possible embodiments and implementations.

In addition to the features mentioned in each of the independent aspects enumerated above, some examples may show, alone or in combination, the optional features mentioned in the dependent aspects and/or as disclosed in the description above and shown in the figures.

1. A suturable cuff for integrating a tissue construct in vivo with a host organ or vasculature, the suturable cuff comprising:
a hollow body having an anastomotic end, an anchoring end, and one or more lumens, each lumen extending through the hollow body from a proximal opening at the anastomotic end to a distal opening at the anchoring end, the anastomotic end being configured for integration with one or more body vessels, and the anchoring end including one or more anchoring features for connection with a tissue construct.

2-3. (canceled)

4. The suturable cuff of claim 1, wherein the one or more anchoring features include one or more springs or coils, one or more harpoons, one or more prongs, one or more struts, one or more rings, and/or one or more mesh structures.

5-6. (canceled)

7. The suturable cuff of claim 1, wherein the one or more anchoring features are bioresorbable.

8. The suturable cuff of claim 1, wherein the one or more anchoring features are coated with one or more molecules to promote adhesion to the tissue construct, the one or more molecules being selected from the group consisting of: polylactic acid, chitosan, alginate, silanes functionalized with N-hydroxysuccinimide, and aldehydes.

9. The suturable cuff of claim 1, further comprising a buffer hydrogel positioned at the anchoring end to promote adhesion to the tissue construct.

10-13. (canceled)

14. The suturable cuff of claim 1, wherein the hollow body comprises a natural or synthetic polymer.

15. (canceled)

16. The suturable cuff of claim 1, wherein the hollow body comprises an extracellular matrix protein or material selected from the group consisting of collagen, fibrin, fibronectin, gelatin, and elastin.

17. The suturable cuff of claim 1, further comprising one or more catheter liners contained within the hollow body, each catheter liner extending from the distal opening to the proximal opening of the hollow body and surrounding one of the lumens,
wherein the one or more catheter liners comprise polytetrafluoroethylene (PTFE), expanded PTFE, polyethylene terephthalate, polyurethane, poly(glycolic acid), polylactic acid, and/or poly lactic-co-glycolic acid.

18. (canceled)

19. The suturable cuff of claim 17, wherein the one or more catheter liners include on inner surface(s) thereof a hydrophilic or zwitterionic polymer, an anti-coagulant drug, an anti-inflammatory agent, an anti-fibrotic agent, an extracellular matrix protein or material, and/or endothelial cells.

20-22. (canceled)

23. The suturable cuff of claim 17, wherein, prior to integration with the hollow body, the one or more catheter liners are etched on outer surface(s) thereof to promote adhesion to the hollow body.

24. The suturable cuff of claim 1, wherein the anastomotic end of the hollow body includes tubular or conical projections, and wherein each lumen is contained within one of the tubular or conical projections.

25-30. (canceled)

31. The suturable cuff of claim 1, wherein the one or more lumens and/or an outer surface of the hollow body includes a functional coating comprising a hydrophilic or zwitterionic polymer, an anti-coagulant drug, an anti-inflammatory agent, an anti-fibrotic agent, an extracellular matrix protein or material, and/or endothelial cells.

32-36. (canceled)

37. A method of integrating a tissue construct with a host organ or vasculature, the method comprising:
providing a suturable cuff comprising a hollow body having an anastomotic end, an anchoring end, and one or more lumens, each lumen extending through the hollow body from a proximal opening at the anastomotic end to a distal opening at the anchoring end;
anchoring a tissue construct including a vascular channel to the anchoring end of the suturable cuff, the vascular channel being positioned to be in fluid communication with the distal opening(s);
inserting the suturable cuff with anchored tissue construct into a body of a patient;
anastomosing one or more body vessels to the anastomotic end of the suturable cuff, whereby the one or more body vessels are in fluid communication with the proximal opening(s); and
perfusing the suturable cuff and the anchored tissue construct with blood or another bodily fluid from the patient.

38. (canceled)

39. The method of claim 37, wherein the anchoring end of the suturable cuff includes one or more anchoring features, and wherein anchoring the tissue construct to the anchoring end of the suturable cuff comprises embedding the one or more anchoring features into the tissue construct.

40. The method of claim 39, wherein embedding the one or more anchoring features within the tissue construct comprises:
preparing a tissue or organ precursor material comprising stem cells mixed with an extracellular matrix solution;
embedding the one or more anchoring features at the anchoring end of the suturable cuff into the tissue or organ precursor material;
moving a nozzle along a print path through the tissue or organ precursor material while a filament comprising a sacrificial ink is extruded through the printing nozzle and deposited along the print path;
after deposition of the filament, heating the tissue or organ precursor material to a temperature at which the precursor material stiffens to form the tissue construct and the sacrificial ink liquifies for extraction; and
removing the sacrificial ink from the tissue construct to form, within the tissue construct, a vascular channel in fluid communication with the distal opening(s) of the suturable cuff.

41. The method of claim 40, further comprising, before embedding the anchoring end of the suturable cuff into the tissue or organ precursor material, compacting the tissue or organ precursor material to increase cellular density.

42. The method of claim 40, further comprising, before moving the nozzle through the tissue or organ precursor material, cooling the tissue or organ precursor material to a temperature at or below 4° C.

43-45. (canceled)

46. The method of claim 37, further comprising seeding the vascular channel with endothelial cells.

47. The method of claim 37, wherein anastomosing the body vessel to the anastomotic end comprises suturing the body vessel to the anastomotic end via an end-to-end connection or a side-to-end T connection.

48. A method of making a suturable cuff, the method comprising:
providing a mold having a cavity shaped to form an exterior of a hollow body, the cavity comprising an upper cavity portion and a lower cavity portion;
filling the lower cavity portion with a precursor material;
situating one or more anchoring features at an end of the lower cavity portion at a cavity location shaped to form part of an anchoring end of the hollow body;
situating one or more rods in the lower cavity portion at one or more cavity regions configured to define one or more lumens of the hollow body, wherein the precursor material in the lower cavity portion at least partly surrounds the one or more anchoring features and the one or more rods;
placing the upper cavity portion over the lower cavity portion to close the mold;
curing the precursor material to form a polymeric body surrounding the one or more rods and part of the one or more anchoring features; and
opening the mold and removing the one or more rods, thereby forming a hollow body comprising the one or more anchoring features, and extracting the hollow body from the mold for use as a suturable cuff.
